(19) United States
(12) Patent Application Publication
(10) Pub. No.: US 2013/0109888 A1
(43) Pub. Date: May 2, 2013

(54) DME-FPSO SYSTEM FOR CONVERSION OF ASSOCIATED GAS IN OIL FIELDS AND STRANDED GAS IN STRANDED GAS FIELDS, AND PROCESS FOR PRODUCTION OF DIMETHYL ETHER USING THE SAME

(71) Applicants: Dong Ju MOON, Seoul (KR); Sang Deuk LEE, Seoul (KR); Byung Gwon LEE, Seoul (KR); Hyun joo LEE, Gyeonggi-Do (KR); Yun Ju LEE, Incheon (KR); Jung Shik KANG, Seoul (KR); Sung Geun LEE, Seoul (KR); Eun Bae LEE, Seoul (KR); Hyun Jin KIM, Gwangju (KR); Bang Hee KIM, Seoul (KR); Jang Jin KIM, Ulsan (KR); Chang Hwan MOON, Busan (KR); Seong Hui HONG, Busan (KR)

(72) Inventors: Dong Ju MOON, Seoul (KR); Sang Deuk LEE, Seoul (KR); Byung Gwon LEE, Seoul (KR); Hyun joo LEE, Gyeonggi-Do (KR); Yun Ju LEE, Incheon (KR); Jung Shik KANG, Seoul (KR); Sung Geun LEE, Seoul (KR); Eun Bae LEE, Seoul (KR); Hyun Jin KIM, Gwangju (KR); Bang Hee KIM, Seoul (KR); Jang Jin KIM, Ulsan (KR); Chang Hwan MOON, Busan (KR); Seong Hui HONG, Busan (KR)

(73) Assignees: KOREA INSTITUTE OF SCIENCE AND TECHNOLOGY, Seoul (KR); DAEWOO SHIPBUILDING & MARINE ENGINEERING CO., LTD., Seoul (KR); DONGHWA ENTEC CO., LTD., Busan (KR); SUNG JIN GEOTEC CO., LTD., Ulsan (KR); HY-LOK CORPORATION, Busan (KR); JNK HEATERS CO., LTD., Seoul (KR)

(21) Appl. No.: 13/285,424

(22) Filed: Oct. 31, 2011

(51) Int. Cl. C07C 41/01; B01J 8/00

(52) U.S. Cl. 568/671; 422/187

(57) ABSTRACT

Disclosed are a dimethyl ether (DME)-floating, production, storage and offloading (FPSO) system that can be used in offshore oil fields or stranded gas fields and a method for producing dimethyl ether using the same. More particularly, the disclosure relates to a DME-FPSO system capable of producing dimethyl ether from gas extracted from stranded gas fields or from associated gas extracted from oil fields, which includes a reforming reactor and a dimethyl ether reactor equipped offshore, and a method for producing the same.

### BACKGROUND

(a) Technical Field

The present invention relates to a dimethyl ether (DME)-floating, production, storage and offloading (FPSO) system that can be used in offshore oil fields or stranded gas fields, and a method for producing dimethyl ether using the same. More particularly, the present invention relates to a DME-FPSO system capable of producing dimethyl ether from gas extracted from stranded gas fields or from associated gas extracted from oil fields, which includes a reforming reactor and a dimethyl ether reactor equipped offshore, and a method for producing the same.

(b) Background Art

With the recent rapid rise in oil price, there has been a growing interest in the use of alternative energy. In particular, the production of synthetic oil from natural gas buried in stranded gas fields as new energy resource has been receiving much attention.

The reforming for producing synthetic gas is mainly achieved through reforming of methane, the major constituent of natural gas. The reforming reactions are largely classified into steam reforming, partial oxidation, autothermal reforming, carbon dioxide reforming, steam carbon dioxide reforming, or the like.

Steam reforming is a method of producing hydrogen-rich synthetic gas via direct contact between methane and steam in the presence of a catalyst. The chemical reaction formula of the steam reforming is as follows:

CH₄+H₂O→3H₂+CO ΔH=226 kJ/mol (1)

As indicated in the above reaction formula, steam reforming is an endothermic reaction and requires a supply of thermal energy from outside.

Unlike the steam reforming, partial oxidation is a method of producing synthetic gas by supplying oxygen. It is divided into non-catalytic partial oxidation and catalytic partial oxidation depending on the use of a catalyst. Because oxygen is needed for the process, investment cost for the oxygen plant facility is high. Further, cokes are generated as byproducts because the reaction is performed at high temperature. The chemical reaction formula of the partial oxidation is as follows:

CH₄+½ O₂→2H₂+CO ΔH=−44 kJ/mol (2)

In autothermal reforming, steam reforming and partial oxidation occur at the same time. The chemical reaction formula is as follows:

CH₄+½ O₂+H₂O→3H₂+CO₂ ΔH=−18 kJ/mol (3)

Carbon dioxide reforming is a method of producing synthetic gas by chemically reacting methane with carbon dioxide. The reaction formula is as follows:

CH₄+CO₂→2H₂+2CO ΔH=261 kJ/mol (4)

Steam carbon dioxide reforming is a method of producing synthetic gas by chemically reacting methane with steam and carbon dioxide. The reactions of the reaction formulae (1) and (4) occur at the same time.

Tri-reforming is a process of producing synthetic gas by chemically reacting methane with steam, carbon dioxide and oxygen. The reactions indicated by formulae (1), (2) and (4) occur at the same time.

Since the facility for producing the synthetic gas accounts for a large portion of investment cost of the plant, selection of an appropriate production method is made in consideration of source materials, scales of the facility and other technical aspects in order to reduce the cost.

Two methods are available for production of dimethyl ether from synthetic fuel using hydrogen and carbon monoxide as follows:

(1-step process) 3H₂+3CO→CH₃OCH₃+CO₂ (5)

(2-step process) 4H₂+2CO→2CH₃OH→CH₃OCH₃+H₂O (6)

In the method based on the reaction formula (5), DME is produced directly using a synthetic gas whose molar ratio of H₂ to CO is adjusted to 1. And, in method based on the reaction formula (6), a synthetic gas whose molar ratio of H₂ to CO is 2 is prepared into methanol first, and then dimethyl ether is prepared from the same through dehydration.

Floating, production, storage and offloading (FPSO) refers to a floating system for producing and storing oil and gas and then offloading to a transportation system such as an oil tanker.

The FPSO includes an apparatus for drilling crude oil and an oil/gas separating apparatus for separating glassy oil into crude oil and associated gas. Further, the FPSO includes a storage apparatus for storing the crude oil and an offloading apparatus for transporting the crude oil.

Recently, a self-powered FPSO system is used because of the necessity for moving to perform the production of crude oil.

The associated gas produced in the FPSO process is either flared and then released to the atmosphere or compressed and then re-injected into the oil wells. Thus, GTL-FPSO or DME-FPSO may be considered to utilize the associated gas from the oil fields as a raw material of the gas to liquid (GTL) process. The natural gas directly extracted from the stranded gas fields may be converted into a synthetic fuel via a GTL-FPSO process or may be directly liquefied via an LNG-FPSO process.

Accordingly, it is important to develop a cost-effective process to utilize the stranded gas from medium-to-small sized stranded gas fields of about 0.1 to 5 Tcf, which account for the majority of gas reserves. In particular, there is an urgent need for the development of a compact process, which is so economical as to recover the investment cost for converting natural gas to a synthetic fuel.

### SUMMARY

The inventors of the present invention have developed a dimethyl ether (DME)-floating, production, storage and offloading (FPSO) system for converting associated gas from offshore oil fields or natural gas from stranded gas fields and a method for producing dimethyl ether using the same. The DME-FPSO system includes a reforming reactor for producing synthetic fuel from associated gas from oil fields and natural gas from stranded gas fields offshore, a dimethyl ether reactor and an internal power generator. A hydrogen separator and a carbon dioxide separation unit are provided between the reforming reactor and the dimethyl ether reactor, and a water separator or a carbon dioxide separator is connected to the dimethyl ether reactor, such that the separated water or carbon dioxide and the water and carbon dioxide produced from the power generator are recycled into the reforming reactor.

In one general aspect, the present invention provides a DME-FPSO system for offshore oil fields, including: an FPSO facility including a glassy oil separator and an oil/gas separation unit; a reforming reactor; a dimethyl ether reactor; a subsea carbon dioxide storage; and an internal power generator, wherein a hydrogen separator and a carbon dioxide separation unit are provided between the reforming reactor and the dimethyl ether reactor and a carbon dioxide separator is coupled to the dimethyl ether reactor, such that separated carbon dioxide, and water and carbon dioxide produced by the internal power generator are recycled to the reforming reactor.

In another general aspect, the present invention provides a DME-FPSO system for offshore stranded gas fields, including: an FPSO facility; a reforming reactor; a dimethyl ether reactor; a subsea carbon dioxide storage; and an internal power generator, wherein a hydrogen separator and a carbon dioxide separation unit are provided between the reforming reactor and the dimethyl ether reactor and a water separator or a carbon dioxide separator is coupled to the dimethyl ether reactor, such that separated water or carbon dioxide, and water and carbon dioxide produced by the internal power generator are recycled to the reforming reactor.

The DME-FPSO system may include a dimethyl ether reactor capable of producing dimethyl ether via a direct method according to the reaction formula (5). Alternatively, it may include a methanol reactor and a dimethyl ether reactor for producing methanol first and then producing dimethyl ether via an indirect method according to the reaction formula (6).

(1-step process) 3H₂+3CO→CH₃OCH₃+CO₂ (5)

(2-step process) 4H₂+2CO→2CH₃OH→CH₃OCH₃+H₂O (6)

The internal power generator may be a polymer electrolyte membrane fuel cell, a solid oxide fuel cell or a molten carbonate fuel cell.

And, the reforming reactor may be a steam reforming reactor, a partial oxidation reactor, an autothermal reforming reactor, a carbon dioxide reforming reactor, a steam carbon dioxide reforming reactor, a tri-reforming reactor, or the like, which is made compact according to the purpose of the DME-FPSO system. In particular, it may be a compact steam reforming reactor, steam carbon dioxide reforming reactor or autothermal reforming reactor.

In another general aspect, the present invention provides a method for producing dimethyl ether using the DME-FPSO system for offshore oil fields, which includes: separating crude oil and gas at an FPSO facility and storing the separated crude oil in a crude oil storage; pretreating the separated gas by saturation and desulfurization; reforming the saturated and desulfurized gas with carbon dioxide and steam to produce a synthetic gas comprising carbon monoxide and hydrogen; removing carbon dioxide from the synthetic gas and returning the removed carbon dioxide to be used as a reaction source of the reforming; chemically reacting the synthetic gas with carbon dioxide removed by the reaction formula (5) or (6) to produce dimethyl ether, and separating the carbon dioxide from the reaction formula (5) and returning the same to be used as a reaction source of the reforming, or producing steam using the water produced by the reaction formula (6) as cooling water for removing the reaction heat and returning the steam to be used in the reforming; producing electric power by operating a fuel cell using the synthetic gas or hydrogen, with water and carbon dioxide being produced during the process; and producing steam using the water produced by the fuel cell as cooling water and returning the same to a reforming reactor along with the produced carbon dioxide:

3H₂+3CO→CH₃OCH₃+CO₂ (5)

4H₂+2CO→2CH₃OH→CH₃OCH₃+H₂O (6)

As in the reaction formula (5), dimethyl ether can be directly synthesized from synthetic gas using a mixture of a synthesis catalyst and a dehydration acid catalyst. The dimethyl ether synthesis using the catalyst mixture provides improved catalytic performance as well as enhanced carbon monoxide conversion and dimethyl ether yield since the product methanol is removed by dehydration and water produced by the dehydration is removed via a water-gas shift reaction. That is to say, when dimethyl ether is synthesized directly from the synthetic gas, three reactions (methanol synthesis, water-gas shift reaction and dehydration) occur simultaneously on the catalyst mixture. The three reactions are complementary and result in a synergic effect.

For the methanol synthesis catalyst, a copper-based ternary catalyst including zinc, alumina, chromium or titanium to modify the support as well as oxide at various proportions. And, an acid catalyst is generally used for the dehydration of methanol. Examples include alumina, zeolite, silica/alumina, metal salt, ion-exchange resin, metal oxide mixture, etc. Also, a catalyst synthesized by coprecipitating the active components may be used. In addition, a catalyst wherein rhodium and molybdenum are supported on y-alumina support, a catalyst prepared by immersing y-alumina in copper nitrate and zinc nitrate solutions, or a catalyst prepared supporting titania on y-alumina may be used.

In another general aspect, the present invention provides a method for producing dimethyl ether using the DME-FPSO system for offshore stranded gas fields, which includes: pretreating stranded gas by saturation and desulfurization; reforming the saturated and desulfurized gas with carbon dioxide and steam to produce a synthetic gas comprising carbon monoxide and hydrogen; removing carbon dioxide from the synthetic gas and returning the removed carbon dioxide to be used as a reaction source of the reforming; chemically reacting the synthetic gas with carbon dioxide removed by the reaction formula (5) or (6) to produce dimethyl ether, and separating the carbon dioxide from the reaction formula (5) and returning the same to be used as a reaction source of the reforming, or producing steam using the water produced by the reaction formula (6) as cooling water for removing the reaction heat and returning the steam to be used in the reforming; producing electric power by operating a fuel cell using the synthetic gas or hydrogen, with water and carbon dioxide being produced during the process; and producing steam using the water produced by the fuel cell as cooling water and returning the same to a reforming reactor along with the produced carbon dioxide:

3H₂+3CO→CH₃OCH₃+CO₂ (5)

4H₂+2CO→2CH₃OH→CH₃OCH₃+H₂O (6)

The composition of the synthetic gas may be adjusted via a water-gas shift reaction or a reverse water-gas shift reaction. Through this, the yield of dimethyl ether may be improved.

In another general aspect, the present invention provides an MeOH-FPSO system for offshore oil fields or stranded gas fields, which includes only the methanol reactor of the DME-FPSO system for offshore oil fields or stranded gas fields, without the dimethyl ether reactor, in order to produce and separate only methanol, which is the intermediate product in the indirect method.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 shows a perspective view and a partial cross-sectional view of a compact steam carbon dioxide reforming reactor according to an embodiment;

FIG. 2 shows a process of producing dimethyl ether using a DME-FPSO system for offshore oil fields via a direct method according to an embodiment; and

FIG. 3 shows a process of producing dimethyl ether using a DME-FPSO system for stranded gas fields via an indirect method according to an embodiment.

[Detailed Description of Main Elements]

| | |
|---|---|
| 200: separator | 201: desulfurizer |
| 202: prereformer | 203: reforming reactor |
| 204: water separator | 205: carbon dioxide removal unit |
| 206: dimethyl ether reactor | 207: gas/liquid separator |
| 208: water separator | 209: fuel separator |
| 210: oil storage | 211: water filter |
| 212: CO₂ storage | 213: DME fuel storage |
| 214: MeOH fuel storage | |
| 300: separator | 301: desulfurizer |
| 302: prereformer | 303: reforming reactor |
| 304: water separator | 305: carbon dioxide removal unit |
| 306: methanol reactor | 307: dimethyl ether reactor |
| 308: gas/liquid separator | 309: water separator |
| 310: fuel separator | 311: oil storage |
| 312: water filter | 313: CO₂ storage |
| 314: DME fuel storage | 315: MeOH fuel storage |

It should be understood that the appended drawings are not necessarily to scale, presenting a somewhat simplified representation of various preferred features illustrative of the basic principles of the invention. The specific design features of the invention as disclosed herein, including, for example, specific dimensions, orientations, locations and shapes, will be determined in part by the particular intended application and use environment.

In the figures, reference numerals refer to the same or equivalent parts of the disclosure throughout the several figures of the drawings.

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The present invention provides a DME-FPSO system producing dimethyl ether from glassy oil or gas extracted from offshore oil fields or stranded gas fields using a reforming reactor, a dimethyl ether reactor, or the like, and a method for producing the same.

The DME-FPSO system mainly comprises a pretreatment apparatus pretreating natural gas, a compact reforming reactor producing synthetic gas, a dimethyl ether reactor producing dimethyl ether from the synthetic gas, a carbon dioxide separation unit for the synthesis of dimethyl ether, a subsea carbon dioxide storage, and an internal power generator for producing electric power required by a water-gas shift reactor or a reverse water-gas shift reactor.

In particular, since the reforming reactor and the dimethyl ether reactor occupying large volume in the DME-FPSO system of the present invention involve exothermic and endothermic reactions, respectively, optimization of the system considering the reactors is required.

In the present invention, in order to perform continuous heat exchange through repeated chemical reactions, a compact fixed-bed type heat exchanger is equipped at the reforming reactor to make the system compact.

In the DME-FPSO system of the present invention, separation of hydrogen from the synthetic gas may be achieved by membrane separation, pressure swing adsorption, cryogenic separation or absorption. Specifically, membrane separation or pressure swing adsorption may be employed.

And, separation of carbon dioxide from the synthetic gas may be achieved by pressure swing adsorption, absorption, cryogenic separation, membrane separation, hybrid separation, or the like. Specifically, membrane separation, pressure swing adsorption or hybrid separation may be employed.

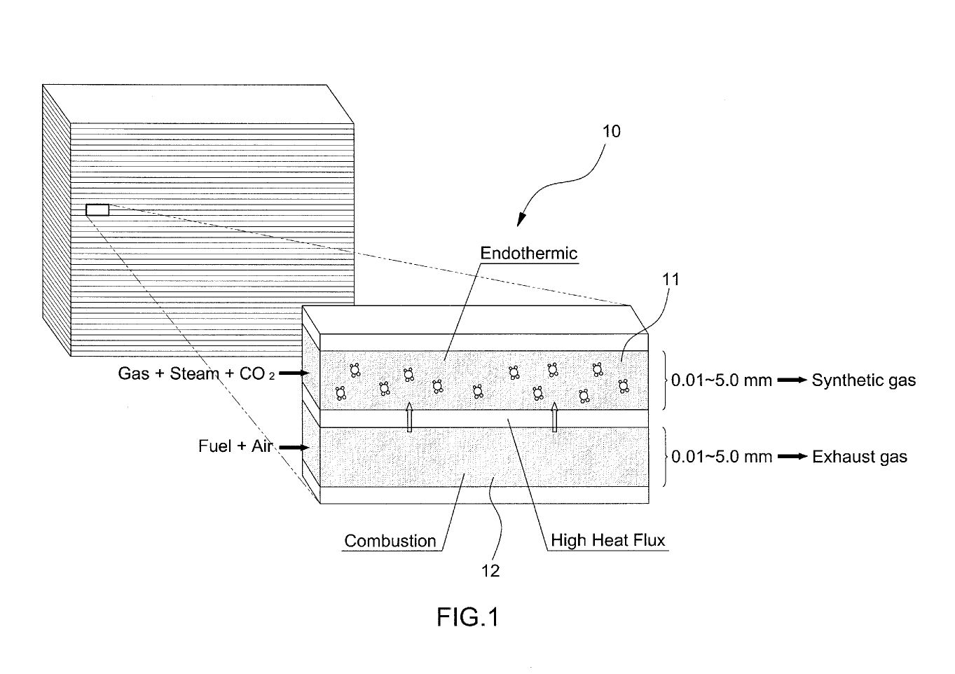

FIG. 1 shows a microchannel type steam carbon dioxide reforming reactor as an example of a compact fixed-bed type reforming reactor for improving reforming efficiency according to an embodiment of the present invention.

The microchannel type reactor is a small reactor including a microchannel heat exchanger configured to improve thermal conductivity and maximize reactor performance. The microchannel type reactor has a plurality of metal plates formed as channels. That is to say, channels formed by etching a plurality of metal plates are arranged perpendicular or parallel to each other, so that fluid may flow through while allowing catalytic reactions and heat exchange at the same time.

The reactions occurring at the microchannel type steam carbon dioxide reforming reactor 10 are as follows:

CH₄+H₂O→3H₂+CO ΔH=226 kJ/mol (1)

CH₄+CO₂→2H₂+2CO ΔH=261 kJ/mol (4)

As seen from the reaction formulae (1) and (4), since the reactions of producing the synthetic gas are endothermic reactions, a mixture 11 comprising a methane-containing gas, steam and carbon dioxide is provided into a thin-walled space (layer), so that heat required for the reaction may be provided effectively. And, a mixture 12 of fuel and air is provided into another layer, so that the heat produced from the combustion of the fuel and air may be provided effectively to the former layer. As illustrated in FIG. 1, as the mixture for reforming the natural gas is provided at high speed and the heat produced from the combustion of the fuel and air is supplied thereto, synthetic gas is produced through the chemical reactions such as those of the reaction formulae (1) and (4), and the fuel and air are discharged as exhaust gas after the combustion. As shown in FIG. 1, the reforming reactor is composed of several layers, each having a width of 0.01 to 10 mm. Since a more effective heat transfer is possible as compared to the existing tube or plate type reactors, the reactor size can be reduced considerably

The DME-FPSO system according to the present invention is equipped with an internal power generator. The power generator may also be a gas turbine or a steam turbine. Specifically, it may be a fuel cell device. Water and CO₂ produced through redox reactions in the course of generating electric power by the fuel cell device may be provided to the reforming reactor to improve the yield of liquid hydrocarbon.

Accordingly, the DME-FPSO system according to the present invention may comprise a fuel cell device capable of producing water and carbon dioxide, which are used as reactants in the reforming reaction.

The fuel cell device used in the present invention may be a polymer electrolyte membrane fuel cell (PEMFC), a solid oxide fuel cell (SOFC) or a molten carbonate fuel cell (MCFC). The reactions occurring in a molten carbonate fuel cell are as follows:

Anode: H₂+CO₃²⁻→2H₂O+CO₂+2e− (7)

Cathode: ½ O₂+CO₂+2e⁻→CO₃²⁻ (8)

The water produced at the anode as byproduct while the electric energy is produced from the fuel cell may be used as cooling water in the DME-FPSO system, and the steam resulting therefrom after heat exchange may be provided to the reforming reactor along with the carbon dioxide also produced at the anode. In this case, the carbon dioxide required in the reaction at the cathode may be supplied partly from the carbon dioxide produced through the reforming and dimethyl ether synthesis reactions and the carbon dioxide produced at the anode.

As another example, the fuel cell device may be a solid oxide fuel cell. The reactions occurring in the solid oxide fuel cell are as follows:

Anode: H₂+O²⁻→H₂O+2e⁻, or (9)

CO+O²⁻→CO₂+2e⁻, or (10)

CH₄+4O²⁻→2H₂O+CO₂+8e⁻ (11)

Cathode: ½ O₂+2e⁻→O²⁻ (12)

As in the molten carbonate fuel cell, the water produced as byproduct while the electric energy is produced from the solid oxide fuel cell may be used as cooling water in the DME-FPSO system, and the steam resulting therefrom after heat exchange may be provided to the reforming reactor. And, carbon monoxide remaining after the dimethyl ether synthesis reaction or carbon dioxide produced from reforming of methane may be recycled to the reforming reactor to improve the efficiency of electric power generation by the fuel cell.

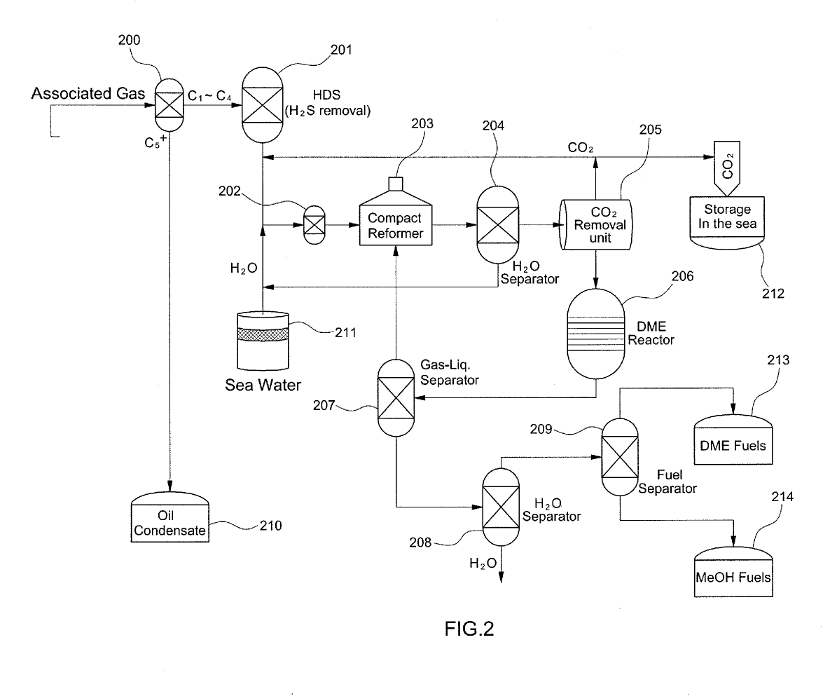

FIG. 2 shows a process of producing dimethyl ether using a DME-FPSO system according to an embodiment.

As shown in FIG. 2, the DME-FPSO system comprises a saturator, a hydrogenation desulfurizer, a compact reforming reactor and a dimethyl ether reactor, as well as separators for separating carbon dioxide, water and the product.

Stranded gas extracted from oil fields is separated by a separator 200 into compounds with 5 or more carbon atoms and those with 1 to 4 carbon atoms. The compounds having 5 or more carbon atoms are condensed and stored in a storage 210, and the compounds having 1 to 4 carbon atoms are, after impurities being removed by a gas desulfurizer 201, passed through a prereformer 202 and provided to a compact reforming reactor 203 for reforming.

Thus produced synthetic gas is a mixture of carbon monoxide and hydrogen. It may be reacted with carbon monoxide and water via a water-gas shift (WGS) reaction as described in the following reaction formula (13) to adjust the composition of CO and H₂.

CO+H₂O [symbol] CO₂+H₂ ΔH (227° C.)=−40 kJ/mol (13)

Also, a reverse water-gas shift reaction process may be performed in order to achieve the desired composition of CO and H₂.

After the water-gas shift reaction process, the synthetic gas is transferred to a water separator 204 and a carbon dioxide removal unit 205 for separation of water and carbon dioxide produced as byproduct. Part of the water separated by the water separator and part of the carbon dioxide separated by the carbon dioxide removal unit may be returned the steam carbon dioxide reforming reaction and to the compact reforming reactor, respectively, to improve the system efficiency. Surplus CO₂ is stored in the sea in wasted gas fields or oil fields 212. After carbon dioxide (CO₂) is removed, the synthetic gas comprising carbon monoxide (CO) and hydrogen (H₂) is transferred to a dimethyl ether reactor 206 for the dimethyl ether synthesis. After the dimethyl ether synthesis reaction according to the reaction formula (5), unreacted material in gas state is recycled to the reforming reactor 203 and the remaining liquid material is transferred to a water separator 208.

3H₂+3CO→CH₃OCH₃+CO₂ (5)

The gas separated by the gas/liquid separator is recycled to the reforming reactor, and the liquid fuel is separated into dimethyl ether and methanol by a fuel separator 209 and then transferred to a DME fuel storage 213 and MeOH fuel storage 214, respectively.

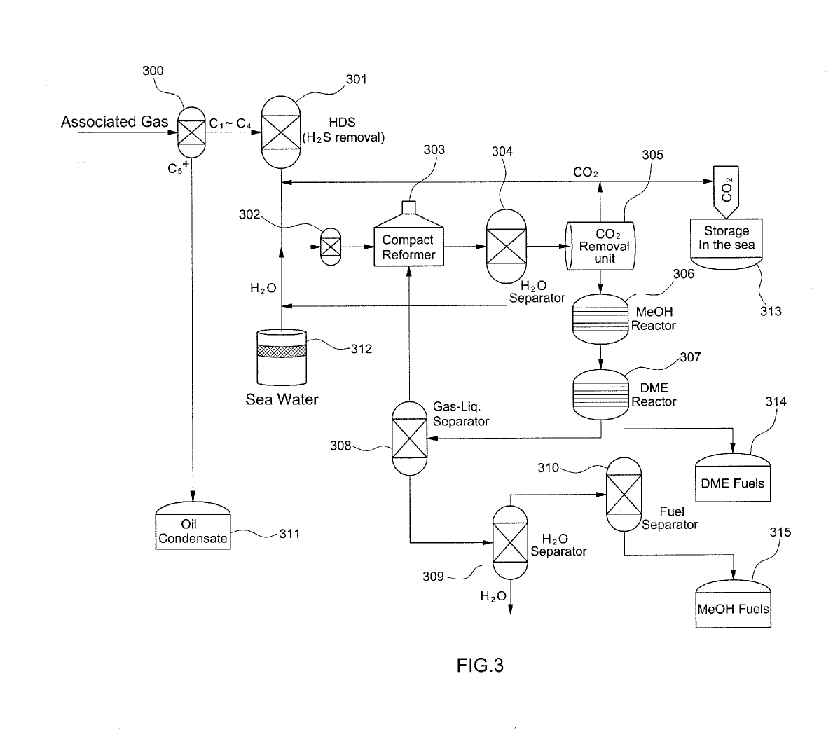

FIG. 3 shows a process of producing dimethyl ether using a DME-FPSO according to another embodiment of the present invention. Dimethyl ether is synthesized via a 2-step process as described in the reaction formula (6). Accordingly, a methanol reactor may be provided in front of the dimethyl ether reactor, and water is produced by the synthesis reaction.

4H₂+2CO→2CH₃OH→CH₃OCH₃+H₂O (6)

The produced water may be separated by a water separator and used as cooling water for the dimethyl ether reactor. The produced dimethyl ether may be stored in a fuel storage. Also, the methanol produced as intermediate may be separately stored.

A MeOH-FPSO process for producing MeOH from gas from offshore oil fields or stranded gas fields may be established by replacing the DME reactor of FIG. 2 with a MeOH reactor or by removing the DME reactor of FIG. 3.

In accordance with the present invention, CO₂ and H₂O produced as byproducts by the fuel cell and CO₂ and H₂O produced from the dimethyl ether synthesis reaction are recycled as source materials for reforming of the natural gas. Especially, since the H₂O is also used as cooling water for removing the heat produced from the dimethyl ether synthesis reaction and surplus CO₂ is be stored in the sea, the productivity of the dimethyl ether synthesis is improved and the cost of CO₂ disposal is minimized while minimizing the size of the DME-FPSO system.

Further, those skilled in the art will easily appreciate that, after crude oil is obtained from offshore oil fields, associated gas may be converted into clean liquid synthetic fuel using the DME-FPSO system according to the present invention. Likewise, clean liquid synthetic fuel may be obtained from natural gas produced from offshore stranded gas fields using the DME-FPSO system.

The DME-FPSO system for offshore oil fields or stranded gas fields and the method for producing synthetic fuel according to the present invention provide the following advantages.

First, associated gas from oil fields and natural gas from stranded gas fields may be converted into dimethyl ether by the DME-FPSO process without emission of CO₂ into the atmosphere. In addition, since surplus CO₂ resulting from the DME process may be recycled to the reforming reactor or stored in the subsea storage, the cost of CO₂ disposal may be minimized.

Second, since surplus hydrogen or synthetic gas from the DME-FPSO process may be utilized to produce electric power, the power required for the DME-FPSO process for producing clean synthetic fuel may be produced offshore without environmental pollution.

Third, since water or carbon dioxide produced by the fuel cell during the production of electric energy may be recycled to the reforming reactor, the efficiency of the DME process may be improved.

Fourth, CO₂ produced from the fuel cell may be recycled to the reforming reactor as a carbon source for preparation of the synthetic fuel, and water-gas (WGS) reaction or reverse WGS reaction may be performed prior to the dimethyl ether synthesis in order to adjust the composition of the synthetic gas required for the dimethyl ether synthesis.

Fifth, the yield of dimethyl ether may be improved by separating CO₂ produced during the reforming and recycling it to the reforming reactor.

Sixth, since CO₂ produced from the fuel cell may be used as a carbon source of the synthetic fuel and surplus CO₂ may be stored in the subsea storage, climatic change caused by CO₂ emission can be prevented.

Seventh, the compact fixed-bed type reactor, specifically the microchannel type reactor, provides improvement in aspects of space and cost. Further, the designing of the DME-FPSO system may be optimized in consideration of safety issue related with the wave motion of the FPSO facility.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

What is claimed is:

1. A dimethyl ether (DME)-floating, production, storage and offloading (FPSO) system for offshore oil fields, for producing DME via a direct method, comprising: an FPSO facility comprising a glassy oil separator and an oil/gas separation unit; a reforming reactor; a dimethyl ether reactor; a subsea carbon dioxide storage; and an internal power generator, wherein a hydrogen separator and a carbon dioxide separation unit are provided between the reforming reactor and the dimethyl ether reactor and a carbon dioxide separator is coupled to the dimethyl ether reactor, such that separated carbon dioxide, and water and carbon dioxide produced by the internal power generator are recycled to the reforming reactor and surplus carbon dioxide is stored in the sea.

2. A DME-FPSO system for offshore oil fields, for producing DME via an indirect method, comprising: an FPSO facility comprising a glassy oil separator and an oil/gas separation unit; a reforming reactor; a methanol reactor; a dimethyl ether reactor; a subsea carbon dioxide storage; and an internal power generator, wherein a hydrogen separator and a carbon dioxide separation unit are provided between the reforming reactor and the methanol reactor and a water separator is coupled to the dimethyl ether reactor, such that separated water, and water and carbon dioxide produced by the internal power generator are recycled to the reforming reactor and surplus carbon dioxide is stored in the sea.

3. DME-FPSO system for offshore oil fields, for producing DME via a direct method, comprising: an FPSO facility; a reforming reactor; a dimethyl ether reactor; a subsea carbon dioxide storage; and an internal power generator, wherein a hydrogen separator and a carbon dioxide separation unit are provided between the reforming reactor and the dimethyl ether reactor and a carbon dioxide separator is coupled to the dimethyl ether reactor, such that separated carbon dioxide, and water and carbon dioxide produced by the internal power generator are recycled to the reforming reactor and surplus carbon dioxide is stored in the sea.

4. A dimethyl ether (DME)-floating, production, storage and offloading (FPSO) system for offshore oil fields, for producing DME via an indirect method, comprising: an FPSO facility; a reforming reactor; a methanol reactor; a dimethyl ether reactor; a subsea carbon dioxide storage; and an internal power generator, wherein a hydrogen separator and a carbon dioxide separation unit are provided between the reforming reactor and the methanol reactor and a water separator is coupled to the dimethyl ether reactor, such that separated water, and water and carbon dioxide produced by the internal power generator are recycled to the reforming reactor and surplus carbon dioxide is stored in the sea.

5. The DME-FPSO system for offshore oil fields according to claim 1 or 2, wherein the internal power generator is a polymer electrolyte membrane fuel cell, a solid oxide fuel cell or a molten carbonate fuel cell.

6. The DME-FPSO system for offshore stranded gas fields according to claim 3 or 4, wherein the internal power generator is a polymer electrolyte membrane fuel cell, a solid oxide fuel cell or a molten carbonate fuel cell.

7. The DME-FPSO system for offshore oil fields according to claim 1 or 2, wherein the reforming reactor is one or more reactor(s) selected from a steam reforming reactor, a partial oxidation reactor, an autothermal reforming reactor, a carbon dioxide reforming reactor, a steam carbon dioxide reforming reactor and a tri-reforming reactor, the reactor(s) being made compact as a fixed bed.

8. The DME-FPSO system for offshore stranded gas fields according to claim 3 or 4, wherein the reforming reactor is one or more reactor(s) selected from a steam reforming reactor, a partial oxidation reactor, an autothermal reforming reactor, a carbon dioxide reforming reactor, a steam carbon dioxide reforming reactor and a tri-reforming reactor, the reactor(s) being made compact as a fixed bed.

9. A method for producing dimethyl ether using a DME-FPSO system for offshore oil fields, comprising:
separating crude oil and gas at an FPSO facility and storing the separated crude oil in a crude oil storage;
pretreating the separated gas by saturation and desulfurization;
reforming the saturated and desulfurized gas with carbon dioxide and steam to produce a synthetic gas comprising carbon monoxide and hydrogen;
removing carbon dioxide from the synthetic gas and returning the removed carbon dioxide to be used as a reaction source of the reforming;
chemically reacting the synthetic gas with carbon dioxide removed by the reaction formula (5) or (6) to produce dimethyl ether;
producing electric power by operating a fuel cell using the synthetic gas or hydrogen, with water and carbon dioxide being produced during the process; and
producing steam using the water produced by the fuel cell as cooling water and returning the same to a reforming reactor along with the produced carbon dioxide:
3H₂+3CO→CH₃OCH₃+CO₂ (5)
4H₂+2CO→2CH₃OH→CH₃OCH₃+H₂O (6)

10. The method for producing dimethyl ether using a DME-FPSO system for offshore oil fields according to claim 9, which further comprises separating the carbon dioxide from the reaction formula (5) and returning the same to be used as a reaction source of the reforming, or producing steam using the water produced by the reaction formula (6) as cooling water for removing the reaction heat and returning the steam to be used in the reforming.

11. The method for producing dimethyl ether using a DME-FPSO system for offshore oil fields according to claim 9 or 10, which comprises adjusting the composition of the synthetic gas required for the production of dimethyl ether via a water-gas shift reaction or a reverse water-gas shift reaction.

12. A method for producing dimethyl ether using a DME-FPSO system for offshore stranded gas fields, comprising:
pretreating stranded gas by saturation and desulfurization;
reforming the saturated and desulfurized gas with carbon dioxide and steam to produce a synthetic gas comprising carbon monoxide and hydrogen;
removing carbon dioxide from the synthetic gas and returning the removed carbon dioxide to be used as a reaction source of the reforming;
chemically reacting the synthetic gas with carbon dioxide removed by the reaction formula (5) or (6) to produce dimethyl ether;
producing electric power by operating a fuel cell using the synthetic gas or hydrogen, with water and carbon dioxide being produced during the process; and
producing steam using the water produced by the fuel cell as cooling water and returning the same to a reforming reactor along with the produced carbon dioxide:
3H₂+3CO→CH₃OCH₃+CO₂ (5)
4H₂+2CO→2CH₃OH→CH₃OCH₃+H₂O (6)

13. The method for producing dimethyl ether using a DME-FPSO system for offshore stranded gas fields according to claim 12, which further comprises separating the carbon dioxide from the reaction formula (5) and returning the same to be used as a reaction source of the reforming, or producing steam using the water produced by the reaction formula (6) as cooling water for removing the reaction heat and returning the steam to be used in the reforming.

14. The method for producing dimethyl ether using a DME-FPSO system for offshore stranded gas fields according to claim 12 or 13, which comprises adjusting the composition of the synthetic gas required for the production of dimethyl ether via a water-gas shift reaction, a reverse water-gas shift reaction or a pressure swing adsorption process.

15. A DME-FPSO system for offshore oil fields, for producing methanol offshore, comprising: an FPSO facility comprising a glassy oil separator and an oil/gas separation unit; a reforming reactor; a methanol reactor; a subsea carbon dioxide storage; and an internal power generator, wherein a hydrogen separator and a carbon dioxide separation unit are provided between the reforming reactor and the methanol reactor and a water separator is coupled to the methanol reactor, such that separated water, and water and carbon dioxide produced by the internal power generator are recycled to the reforming reactor and surplus carbon dioxide is stored in the sea.

16. A DME-FPSO system for offshore stranded gas fields, for producing methanol offshore, comprising: an FPSO facility comp; a reforming reactor; a methanol reactor; a subsea carbon dioxide storage; and an internal power generator, wherein a hydrogen separator and a carbon dioxide separation unit are provided between the reforming reactor and the methanol reactor and a water separator is coupled to the methanol reactor, such that separated water, and water and carbon dioxide produced by the internal power generator are recycled to the reforming reactor and surplus carbon dioxide is stored in the sea.
